(19) United States
(12) Patent Application Publication
(10) Pub. No.: US 2025/0380877 A1
(43) Pub. Date: Dec. 18, 2025

(54) System for determining the optimal time for aortic valve replacement based on Myocardial dysfunction

(71) Applicant: Medici Technologies, LLC, Albuquerque, NM (US)

(72) Inventors: Mark Ries Robinson, Albuquerque, NM (US); Jasmine Sanchez, Albuquerque, NM (US)

(21) Appl. No.: 19/241,074

(22) Filed: Jun. 17, 2025

Related U.S. Application Data
(60) Provisional application No. 63/660,659, filed on Jun. 17, 2024.

(51) Int. Cl. A61B 5/02; A61B 5/00; A61B 5/0295; A61B 5/11; G16H 15/00

(52) U.S. Cl. CPC A61B 5/02028; A61B 5/0295; A61B 5/1116; A61B 5/6826; A61B 5/7264; A61B 5/7271; G16H 15/00; A61B 2560/0462; A61B 2562/0219; A61B 2562/0238

(57) ABSTRACT

A non-invasive diagnostic system assesses the optimal timing for aortic valve replacement (AVR) by quantifying left-ventricular Frank-Starling reserve before irreversible myocardial damage occurs. The system (i) acquires left-ventricular ejection time (LVET) and other systolic-time intervals from optical or vibrational sensors positioned on the patient, (ii) induces a reversible preload change—e.g., passive leg raise or posture transition—to create a controlled venous-return increment, (iii) processes the paired baseline and post-maneuver waveforms to extract systolic time interval metrics, and (iv) analyzes the ΔLVET/Δpreload relationship against historical or population references. A diminished LVET response signals loss of contractile reserve, enabling timely AVR while myocardial changes remain reversible. The platform integrates measurement hardware, a data-processing engine, a data-analysis module, and a reporting interface, and may be configured as a wrist, ring, chest, or ear sensor. The method can be implemented at point-of-care without operator-dependent imaging or invasive monitoring.

### TECHNICAL FIELD

The present invention relates to determining the optimal time for aortic valve replacement by measuring the Frank-Starling reserve. The system provides for a sensitive determination of Frank-Starling reserve of the left ventricular using left ventricular ejection time in the presence of changes in preload to the heart to determine the optimal time for aortic valve replacement. The noninvasive system enables the determination of diminished Frank-Starling reserve prior to the development of significant irreversible myocardial changes, such as fibrosis. Valve replacement before the development of irreversible damage to the heart creates an improved outcome for patients with aortic stenosis. The system enables the assessment of aortic valve stenosis and the concurrent assessment of Frank-Starling reserve in a systematic and straightforward manner.

### BACKGROUND

The management of aortic stenosis (AS) presents significant challenges, particularly in determining the optimal timing for surgical intervention. Aortic stenosis is characterized by the progressive thickening, fibrosis, and calcification of the aortic valve leaflets, which leads to restricted valve opening and increased left ventricular afterload. This pressure overload initially results in a compensatory hypertrophic response of the left ventricle, which helps to maintain cardiac output, and stroke volume. Early left ventricular hypertrophy (LVH), where the heart muscle thickens to compensate for increased pressure, can be partially reversible if treated promptly, such as through valve replacement. However, as the disease progresses, this compensatory mechanism fails, leading to myocardial dysfunction, irreversible damage, and eventually symptomatic heart failure.

In the early stages of AS, the myocardial changes are largely hypertrophic and can be reversed with timely valve replacement. However, if intervention is delayed and aortic stenosis progresses, the degree of hypertrophy advances with the heart muscle becomes excessively thickened, leading to diastolic dysfunction (stiffness and impaired relaxation), systolic dysfunction (reduced pumping ability), and fibrosis (development of fibrous tissue). Myocardial fibrosis and scarring, as well as cardiomyocyte death, are irreversible changes. Thus, the key challenge in managing AS is to accurately identify the point at which myocardial changes transition from being reversible to irreversible.

Conventional echocardiography, while valuable for assessing the severity of aortic stenosis (AS), faces notable challenges in detecting early myocardial dysfunction. Its primary focus on valvular morphology and hemodynamics, such as valve area and transvalvular gradients, often fails to capture the subtle myocardial changes that occur in the early stages of the disease. Additionally, the resolution of standard echocardiographic images is often insufficient to detect early fibrosis, hypertrophy, or other microscopic changes in myocardial tissue. Although techniques like speckle-tracking echocardiography (STE) offer insights into myocardial strain and deformation, they still do not match the detail provided by cardiac MRI or CT. The assessment of diastolic function through echocardiography can be complex and less reliable, particularly in patients with comorbid conditions like hypertension. The accuracy of echocardiography also heavily depends on the operator's skill and experience, leading to variability in detecting subtle myocardial changes. In contrast, advanced imaging modalities like cardiac MRI and CT offer superior spatial resolution and tissue characterization, detecting early myocardial changes with high accuracy. However, these techniques are expensive and less accessible, limiting their routine use. This constraint on resources often means patients are monitored with conventional echocardiography alone, potentially delaying the detection of diminished Frank-Starling reserve and subsequent clinical intervention. By the time dysfunction is evident on echocardiography, significant myocardial damage may have already occurred, affecting patient outcomes. Thus, while conventional echocardiography is a critical tool in AS evaluation, its limitations highlight the need for an improved method that is inexpensive and not operator-dependent.

Current clinical guidelines recommend surgical intervention for severe aortic stenosis (AS) based on the presence of symptoms or evidence of left ventricular decompensation, such as reduced ejection fraction or elevated B-type natriuretic peptide (BNP) levels. These indicators often represent a relatively late stage of myocardial damage and typically require invasive procedures, such as blood draws for BNP measurement or additional echocardiograms, to assess the severity of the condition. Furthermore, evaluating AS severity can be particularly challenging in patients with discordant echocardiographic measurements or those in low-flow states.

The reliance on symptoms to guide intervention in AS is fraught with issues. Symptoms are highly subjective and can vary significantly across individuals. Older individuals, in particular, may confuse symptoms of aging, such as fatigue or decreased exercise tolerance, with those of aortic stenosis, leading to underreporting and misattribution. This variability in symptom perception can result in delayed diagnosis and treatment, further complicating the management of AS.

Everatt et al describe the problem well and state, 37 Timing of valve intervention is crucial. Too early and the patient will be unnecessarily exposed to risks of intervention and prosthetic valve complications; too late and irreversible myocardial damage can lead to persistent symptoms and risk of adverse events. Ideally valve replacement would be performed just as left ventricular decompensation is starting to develop. (Everett, Russell James, et al. “Timing of intervention in aortic stenosis: a review of current and future strategies.” Heart 104.24 (2018): 2067-2076.) FIG. 1 is a diagram from the Everett paper.

Conventional echocardiography, while valuable for assessing the severity of aortic stenosis (AS), faces notable challenges in detecting early myocardial dysfunction. Its primary focus on valvular morphology and hemodynamics, such as valve area and transvalvular gradients, often fails to capture the subtle myocardial changes that occur in the early stages of the disease. Additionally, the resolution of standard echocardiographic images is often insufficient to detect early fibrosis, hypertrophy, or other microscopic changes in myocardial tissue. Although techniques like speckle-tracking echocardiography (STE) offer insights into myocardial strain and deformation, they still do not match the detail provided by cardiac MRI or CT. The assessment of diastolic function through echocardiography can be complex and less reliable, particularly in patients with comorbid conditions like hypertension. The accuracy of echocardiography also heavily depends on the operator's skill and experience, leading to variability in detecting subtle myocardial changes. In contrast, advanced imaging modalities like cardiac MRI and CT offer superior spatial resolution and tissue characterization, detecting early myocardial changes with high accuracy. However, these techniques are expensive and less accessible, limiting their routine use. This constraint on resources often means patients are monitored with conventional echocardiography alone, potentially delaying the detection of early and subsequent clinical intervention. By the time dysfunction is evident on echocardiography, significant myocardial damage may have already occurred, affecting patient outcomes. Thus, while conventional echocardiography is a critical tool in AS evaluation, its limitations highlight the need for an improved method that is inexpensive and not operator dependent.

Recent research increasingly supports the need for earlier aortic valve replacement (AVR) in patients with aortic stenosis (AS), even before the onset of overt symptoms or significant left ventricular dysfunction. Traditional guidelines have prioritized symptom onset and clear echocardiographic evidence of severe AS as triggers for intervention. However, accumulating evidence demonstrates that irreversible myocardial injury, such as fibrosis and subclinical ventricular dysfunction, can develop prior to the appearance of symptoms or notable changes in left ventricular ejection fraction (LVEF) (Kang, D.H., Park, S.J., Lee, S.A., et al. (2020). Early Surgery or Conservative Care for Asymptomatic Aortic Stenosis. New England Journal of Medicine, 382(2), 111-119. and Kang, D.H., Park, S.J., Lee, S.A., et al. (2020). Early Surgery or Conservative Care for Asymptomatic Aortic Stenosis. New England Journal of Medicine, 382(2), 111-119). Several observational studies and meta-analyses have shown that patients undergoing AVR before symptom development or significant ventricular impairment experience lower mortality rates and better long-term outcomes than those who wait until conventional criteria are met (Généreux, P., Stone, G.W., O'Gara, P.T., et al. (2016). Natural History, Diagnostic Approaches, and Therapeutic Strategies for Patients with Asymptomatic Severe Aortic Stenosis. Journal of the American College of Cardiology, 67(19), 2263-2288. and Van Gils, L., Clavel, M.A., Vollema, E.M., et al. (2017). Prognostic Implications of Conventional and Advanced Imaging Markers of Left Ventricular Remodeling in Aortic Stenosis. JACC: Cardiovascular Imaging, 10(10), 1360-1373). Despite this, determining the optimal timing for AVR remains challenging, as there is no single marker that reliably predicts the transition from reversible to irreversible myocardial damage.

A major barrier to optimal timing is the limitation of conventional echocardiography, which remains the primary imaging modality for AS assessment. While echocardiography excels at evaluating valvular anatomy and hemodynamics, it is less sensitive for detecting early or subtle myocardial changes such as diffuse fibrosis or early hypertrophy (Dweck, M.R., Joshi, S., Murigu, T., et al. (2011). Left Ventricular Remodeling and Myocardial Fibrosis in Aortic Stenosis: Insights from Cardiovascular Magnetic Resonance. Journal of the American College of Cardiology, 58(12), 1271-1279). Advanced techniques like speckle-tracking echocardiography (STE) can assess myocardial strain, offering some improvement in detecting early dysfunction, but still fall short of the tissue characterization capabilities of cardiac MRI. The accuracy of echocardiographic assessment is also highly operator-dependent, and diastolic function evaluation can be particularly unreliable in patients with comorbid conditions. In contrast, cardiac MRI provides superior spatial resolution and can directly visualize myocardial fibrosis, but its high cost and limited availability restrict its routine use. As a result, many patients are monitored with echocardiography alone, which may delay the detection of Frank-Starling reserve and, consequently, timely intervention. This underscores the urgent need for more accessible, reliable, and sensitive diagnostic tools to guide AVR timing and improve patient outcomes.

An improved method for determining the optimal time for aortic valve replacement is needed that is inexpensive, simple to use, and can be performed in a variety of care locations, including the primary care clinic or cardiology clinic.

### DISCLOSURE OF INVENTION

Embodiments of the present invention provide an apparatus for determining and assessing Frank-Starling reserve by using LVET measurements in the presence of systematic changes in filling pressure to the heart. The changes in LVET can be quantified relative to population-based changes or to prior measurements on the same patient to detect alterations in cardiac function due to changes in the aortic valve or myocardium.

Embodiments of the present invention enable assessment of the degree of valvular change by examining changes over time in LVET when the heart is exposed to repeatable changes in filling pressure.

Embodiments of the present invention can determine LVET based on PPG or SPG pulse waves obtained in two or more body positions or two or more positions that alter venous return. The change in LVET between body positions resulting in increased filling pressure can be used to detect the onset of myocardial dysfunction. The initiation of diminished Frank-Starling reserve can be used to determine the appropriate time for valve replacement.

Embodiments of the current invention can be combined with a system for the determination of aortic valve area to determine the degree of aortic stenosis. The combination of aortic valve functional status and myocardium status creates a holistic system for the management of valvular disease by providing independent pieces of information at the time of measurement or over time for determining the optimal time for aortic valve replacement.

### BRIEF DESCRIPTION OF DRAWINGS

FIG. 1 is an illustration of the optimal time for valve replacement.

FIG. 2 is the influence of venous return on LVET.

FIG. 3 is a graph showing the influence pf changes in venous return across several subjects.

FIG. 4 is an illustration of LVET changes due to preload changes in presence of AS and normal myocardial function.

FIG. 5 is an illustration of LVET changes due to preload changes in presence of AS and myocardial dysfunction.

FIG. 6 is an illustration of LVET changes due to preload changes in presence of AS and increased myocardial dysfunction.

FIG. 7 is an illustration of LVET changes and FIG. 1 is an illustration of LVET changes due to preload changes in presence of AS and increased myocardial dysfunction.

FIG. 8 is an illustration of example fiducial marks.

FIG. 9, comprising FIGS. 9A, 9B, 9C, 9D, and 9E, provides illustrations of LVET changes due to preload changes in presence of AS and increased myocardial dysfunction.

FIG. 10 is an illustration of passive leg raising maneuvers.

FIG. 11 is an illustration of an example measurement sequence.

FIG. 12 is an illustration of a combined photoplethysmography (PPG) and speckle plethysmography (SPG) measurement system configured for chest-based acquisition of cardiovascular signals.

FIG. 13 is an illustration of an example data pathway.

FIG. 14 is an illustration of a report with prior measurements.

FIG. 15 is an illustration of a report with no prior measurements.

FIG. 16 is an illustration of a report with no prior measurements.

FIG. 17 is an illustration of an example of a blunted Frank-Starling response determinations. FIG. 17 defines a method that could be used to establish physiological thresholds representing the lower bound of a normal Frank-Starling response.

### DEFINITIONS

Noninvasive sensors, as used herein, refers to a class of sensors that can be used outside the body and are sensitive to blood flow and blood volume, cardiac function, and physiological signals.

Electrocardiogram, as used herein, is a test that records the electrical activity of the heart. The measured signals can be used in both physiological assessments and the determination of cardiac fitness.

Phonocardiogram, as used herein, is a recording of the sounds made by the heart and are related to the mechanical activities of the heart. The measured signals can be used in both physiological assessments and the determination of cardiac fitness.

Seismocardiogram, as used herein, is a technique for recording and analyzing cardiac vibratory activity as a measure of cardiac contractile functions. The measured signals can be used in both physiological assessments and the determination of cardiac fitness.

Ballistocardiography, as used herein, is a technique for producing a graphical representation of the reaction of the body to cardiac ejection forces or the reaction of the body to the blood mass ejected by the heart with each contraction associated with arterial circulation. The measured signals can be used in both physiological assessments and the determination of cardiac fitness.

Vibrational and acoustic measures, as used herein, refers to those measurement technologies that are sensitive to the vibration generated by the heart or blood flow and include phonocardiogram, seismocardiogram, ballistocardiography, or any other method that is sensitive to the vibrations or sound created by the heart.

Echocardiography, as used herein, is the use of ultrasound to investigate the action and functioning of the heart. The measured signals can be used for physiological assessments.

Speckle plethysmograph (SPG), as used herein, is a noninvasive optical measurement system that measures blood flow in the body. The system uses a laser or other light source to illuminate the skin and tissue, and then analyzes the scattered light patterns, or speckles, which are produced. The system can operate in reflection sampling mode and transmission sampling mode. The system can be used to measure blood flow in various parts of the body, such as the hand, finger, wrist, foot, or brain, and can provide important information about the function of the circulatory system and the health of tissues and organs. A speckle sensor system generates a plethysmogram that represents changes in blood flow throughout the cardiac cycle.

Photo plethysmograph (PPG), as used herein, is an optical measurement system that measures changes in blood volume using changes in light absorption and can be used to measure blood volume in a transmission sampling mode and a reflection sampling mode. The measured signal is commonly reerd to as a plethysmogram.

Radar plethysmograph (RPG), as used herein, is a noninvasive millimeter-wave, radar-based device for the accurate measurement of arterial pulse waveforms. Radar plethysmography can be utilized at any location on the body where a pulse creates a detectable movement of the skin or tissue. A common location is to use the system as a wrist-worn device that positions the radar near the radial artery without touching the skin, allowing for interrogation of the pulse at close range without perturbing the pulse waveform.

Thoracic bioimpedance (also called impedance cardiography), as used herein, denotes any non-invasive technique in which a small, alternating electrical current is driven through surface electrodes placed on the torso and the resulting beat-to-beat changes in thoracic impedance, produced by pulsatile blood flow and tissue motion, are analyzed to estimate stroke volume, cardiac output, or related hemodynamic indices.

Bio-reactance, as used herein, refers to a variant of thoracic impedance measurement in which the phase shift of the injected alternating current is processed, rather than its amplitude, to derive the same hemodynamic indices, thereby allowing useful estimates even when electrode placement or tissue conductivity varies.

Gyrocardiography, as used herein, means the acquisition and analysis of rotational kinematic signals, typically angular velocity or acceleration, generated by cardiac mechanical activity and sensed by one or more micro-electromechanical gyroscopes placed on the chest or torso; the derived waveform features serve as surrogates for ventricular ejection timing or momentum.

“Doppler patch,” as used herein, denotes a self-contained, skin-adherent ultrasound transducer assembly that transmits and receives continuous-wave or pulsed Doppler signals from an underlying blood vessel or cardiac structure and computes flow-related metrics, such as velocity-time integral or corrected flow time, suitable for trending changes in stroke volume in response to a preload-modifying maneuver.

Optical sensors, as used herein, refers to any optically based system that can be used to capture signals related to changes in blood volume, flow, or pressure in a measurement region of the individual, which changes are indicative of cardiac function.

Systolic time intervals, as used herein, are one or more calculated or measured parameters that describe the temporal phases of the cardiac cycle. Cardiac-specific systolic time intervals include EMAT (electromechanical activation time), ICT (isovolumic contraction time), PEP (pre-ejection time), heart rate, interbeat interval, and LVET (left ventricular ejection time). Parameters associated with pulse transit times are PTT (pulse transit time) and PAT (pulse arrival time).

An alteration in venous return, as used herein, refers to activities that change the filling pressure into the heart in a systematic fashion. Alterations in venous return can be accomplished but are not limited to intrathoracic pressure changes, changes in the total circulating volume, and alterations in the distribution or location of the circulating volume.

“Changes in preload” refers to variations in the degree of myocardial stretch that occurs at the end of ventricular diastole, just before contraction. This stretch is primarily governed by the volume of blood filling the ventricles—particularly the left ventricle—and directly influences stroke volume through the Frank-Starling mechanism. Preload is not a discrete anatomical structure or a single pressure measurement but rather a functional concept describing the loading condition of the heart under a given set of filling dynamics. It is physiologically determined by factors such as venous return, ventricular compliance, intrathoracic pressure, and the distribution of blood within the vascular system. Changes in preload may be acute or sustained and may result from postural changes, intravascular volume shifts, altered vascular tone, or underlying cardiac dysfunction. While “preload” is the preferred physiologic term, related expressions are frequently used to describe similar or overlapping concepts, including “increased venous return” (reflecting more blood returning to the heart), “increased end-diastolic volume” (EDV) or “end-diastolic pressure” (EDP) as direct surrogates of preload, “increased filling pressure” such as central venous pressure (CVP) or pulmonary capillary wedge pressure (PCWP) as clinical approximations, and “volume loading,” which refers more broadly to conditions or interventions that increase preload by augmenting circulating blood volume.

Mechanisms for changing preload” refers to any process, maneuver, or condition that alters venous return to the heart and thereby modifies the volume of blood filling the ventricles at the end of diastole. A key subset of such mechanisms involves positional or postural changes, which can shift blood between central and peripheral compartments due to gravity. For example, moving from a supine to a standing position reduces preload by promoting peripheral pooling of blood, while lying flat or raising the legs increases preload by enhancing central blood volume. A particularly well-characterized clinical maneuver is the Passive Leg Raise (PLR), in which a patient's legs are elevated (typically to 45 degrees) while in a supine or semi-recumbent position. This transiently increases venous return from the lower extremities to the thoracic cavity, effectively creating an “auto-bolus” that simulates fluid administration without infusing actual volume. PLR is widely used as a reversible and reproducible method to test preload responsiveness and assess fluid responsiveness in both critical care and ambulatory settings. Other mechanisms for changing preload may include respiratory maneuvers (e.g., spontaneous breathing, mechanical ventilation, or Valsalva), muscle contractions, pharmacologic interventions, or disease states that affect intravascular volume or vascular capacitance. Pneumatic lower thorax, leg, or calf cuffs can be used to force blood from the lower extremity.

Postural change, as used herein, means a deliberate, gravity-mediated maneuver—such as stand-to-supine transition, passive leg raise, or head-up/head-down tilt—that redistributes venous blood toward or away from the thorax and thereby acutely alters ventricular preload.

Frank-Starling Reserve, as used herein, is the heart's capacity to increase stroke volume in response to acute preload augmentation. This augmentation occurs due to increased preload (end-diastolic pressure), where greater ventricular diastolic volume stretches myocardial fibers during diastole. The increased tension in the muscle fibers increases the force of contraction when stimulated. In healthy individuals, stroke volume or LVET typically increases by ˜15% with preload enhancement that occurs via the a supine to stand postural change. In patients with heart failure, this reserve is depressed or absent, and stroke volume does not change significantly. The mechanism operates through myofilament length-dependent activation, making it a fundamental index of preload-responsive contractile reserve.

Diminished Frank-Starling Reserve, as used herein, exists when the preload-induced rise in stroke volume or its surrogate (e.g., LVET) falls below the range expected from normative population data, demographic-adjusted models, or the subject's own prior baseline under the same preload stimulus. Operationally, a shortfall of ≥10% relative to the anticipated change is considered diminished, indicating loss of preload-dependent contractile reserve.

“Blunted response” as used herein, is any under-performance of a preload-sensitive cardiac metric—stroke volume, cardiac output, LVET, or similar—relative to the change predicted for a heart with preserved Frank-Starling reserve after a defined preload challenge. It is a measured phenomenon rather than a diagnosis, and it reflects reduced capacity to augment function in response to increased venous return.

Data-processing system—any hardware, firmware, software, or cloud module that receives raw sensor data, performs signal conditioning and feature extraction of any suitable kind, and outputs time-synchronized physiological metrics with associated quality indices. Location, architecture, and processing techniques are unrestricted.

Data-analysis system—a module or platform that ingests processed physiological metrics, contextualises them with historical or population reference data, applies threshold or model-based logic, and generates diagnostic or comparative outputs, including confidence measures.

### DETAILED DESCRIPTION OF INVENTION

The invention provides a simple, inexpensive, and easy-to-administer test that identifies the transition from normal myocardium function to dysfunctional myocardium in the presence of aortic stenosis. Typically, individuals with aortic stenosis have an elongated left ventricular ejection time (LVET) because it takes more time to push blood through the narrowed aortic valve opening. Thus, the invention enables the assessment of myocardial damage in the presence of aortic stenosis and in the presence of aortic stenosis progression. The test enables the assessment of Frank-Starling reserve by examining LVET before and after a repeatable change in the preload of the heart.

In a normally functioning myocardium, a passive leg raise increases the preload or venous return, leading to an increased stroke volume and, consequently, a longer LVET. The absence of an increase in LVET in response to this preload change indicates myocardial dysfunction, an abnormal response. This personalized assessment can be established at the time of initial diagnosis of aortic stenosis. This simple but specific test can be used to detect when the myocardium transitions from normal functioning to a degree of dysfunction. The presence of a dysfunctional myocardium is typically associated with the start of irreversible myocardial changes.

The test is personalized, recognizing that factors such as body size and characteristics influence the volume or preload change induced by the passive leg raise. By measuring LVET in both the supine and leg raise positions, the test provides a clear and individualized approach to determining the onset of diminished Frank-Starling reserve and subsequently enables the optimal timing for valve replacement. Monitoring this change over time allows clinicians to identify when the myocardium begins to exhibit decreased capacity, indicating the onset of irreversible damage. A decreased change in LVET following a passive leg raise is a clear indication of the start of diminished Frank-Starling reserve and can be used as a definitive input into the consideration for valve replacement. This system provides a non-invasive, accurate measure to guide the timing of intervention in AS and enables the detection of early myocardial decompensation for improve patient outcomes.

Assessing myocardial changes of the heart with echocardiography can be challenging due to several factors. Echocardiography primarily relies on two-dimensional imaging, which can limit the accuracy and precision in measuring the three-dimensional structure of the myocardium. Variability in image quality, due to patient anatomy, body habitus, or operator skill, can further complicate the assessment. Additionally, echocardiographic measurements are angle-dependent, making it difficult to obtain consistent and reproducible views of the hypertrophied myocardium. Furthermore, differentiating between pathological and reversible hypertrophy caused by increased afterload versus irreversible changes due to fibrosis can be challenging. These limitations can lead to variability in diagnosing the extent and severity of hypertrophy, ultimately affecting clinical decision-making and patient management.

### Understanding the Physiology Behind LVET Changes

LVET is the time between opening of the aortic valve and the closing of the aortic valve. LVET is a time-based measurement of cardiac function and can be obtained via multiple methods including, as examples, PPG and SPG measurements. Photoplethysmography (PPG) is a non-invasive optical technique that measures blood volume changes in the tissue. By detecting variations in light absorption due to pulsatile blood flow, PPG can generate arterial waveforms that are useful in determining left ventricular ejection time (LVET). This allows for continuous and real-time cardiovascular monitoring in both clinical and wearable device settings. Speckle Plethysmography (SPG) is another non-invasive method that uses the speckle pattern produced by coherent light interaction with tissue to capture microvascular blood flow and arterial pulse waveforms. The analysis of temporal fluctuations in the speckle pattern provides detailed information on blood flow dynamics. SPG waveforms can also be used to accurately determine LVET, offering insights into cardiovascular health.

For determining the time for surgical intervention, LVET provides valuable information due to the fact that both AS and diminished Frank-Starling reserve impact LVET. The competing effects of aortic stenosis (AS) and decreased Frank-Starling reserve can be understood through their respective impacts on the heart's hemodynamics.

### Aortic Stenosis and Prolonged LVET

In AS, the aortic valve area becomes progressively smaller, causing an increased resistance to blood flow from the left ventricle into the aorta. As a result, the left ventricle needs more time to eject the blood, leading to a prolonged LVET. This can be explained by the Gorlin formula used to calculate the aortic valve area (AVA), where:

$$AVA = \frac{CO}{LVET \times HR \times 44.3 \times \sqrt{MG}}$$

CO is the cardiac output, HR is the heart rate, MG is the mean transvalvular gradient.

The formula shows that as the aortic valve area (AVA) decreases, indicating more severe aortic stenosis, the resistance to systolic outflow from the left ventricle increases. To maintain forward stroke volume, the left ventricle must generate higher pressure and eject blood more slowly through the narrowed valve. As a result, more time is needed to eject blood through the restricted opening, which leads to a prolongation of the left ventricular ejection time (LVET). This prolonged LVET, assuming heart rate and contractility remain stable, reflects the increased afterload the heart must overcome to maintain effective cardiac output in the setting of aortic stenosis.

### Myocardial Dysfunction and the Shortening of LVET

Myocardial dysfunction, in the absence of aortic stenosis, can lead to a shortening of the left ventricular ejection time (LVET). When the myocardium is weakened, its ability to contract effectively and generate adequate pressure during systole is impaired. As a result, the ejection phase begins later—due to a prolonged isovolumetric contraction—and ends earlier, as the failing myocardium cannot sustain sufficient pressure to maintain forward flow. This abbreviated ejection window leads to a shortened LVET, which reflects the diminished contractile reserve of the heart.

The above physiological mechanisms, when present independently, create a competing effect on LVET. AS tends to prolong LVET as the ventricle needs more time to eject blood through a narrowed valve, while myocardial dysfunction tends to shorten LVET due to reduced contractility.

### Aortic Stenosis and Myocardial Dysfunction

The overall impact on LVET in a patient with both AS and myocardial dysfunction will depend on the relative severity of each condition. In the early stages of AS without significant myocardial dysfunction, LVET may be predominantly prolonged due to the increased ejection resistance. In advanced stages where myocardial dysfunction is significant, the shortening effect due to poor myocardial contractility can counteract the prolongation effect of AS, potentially resulting in a shorter or less prolonged LVET than expected from AS alone.

The assessment of LVET in patients with AS must therefore consider both the degree of valve stenosis and the extent of myocardial dysfunction. The measurement of a single LVET does not enable the assessment of these two conditions. Thus, a single LVET measurement does not enable accurate assessment of AS severity or myocardial dysfunction.

### Functional Testing with Blunted Frank-Starling Responsiveness

A postural change, such as a passive leg raise, represents a repeatable method for increasing venous return. A passive leg raise involves lifting the patient's legs to a position above the level of the heart. This maneuver increases venous return to the heart by shifting blood from the lower extremities to the central circulation. The resulting increase in preload leads to an increase in end-diastolic volume (EDV), which enhances the stroke volume due to the Frank-Starling mechanism. The increased EDV creates an increased stretch of the ventricular myocardium, resulting in an enhanced force of contraction, leading to a higher stroke volume (SV). This increased stroke volume requires a longer ejection phase, thus elongating LVET.

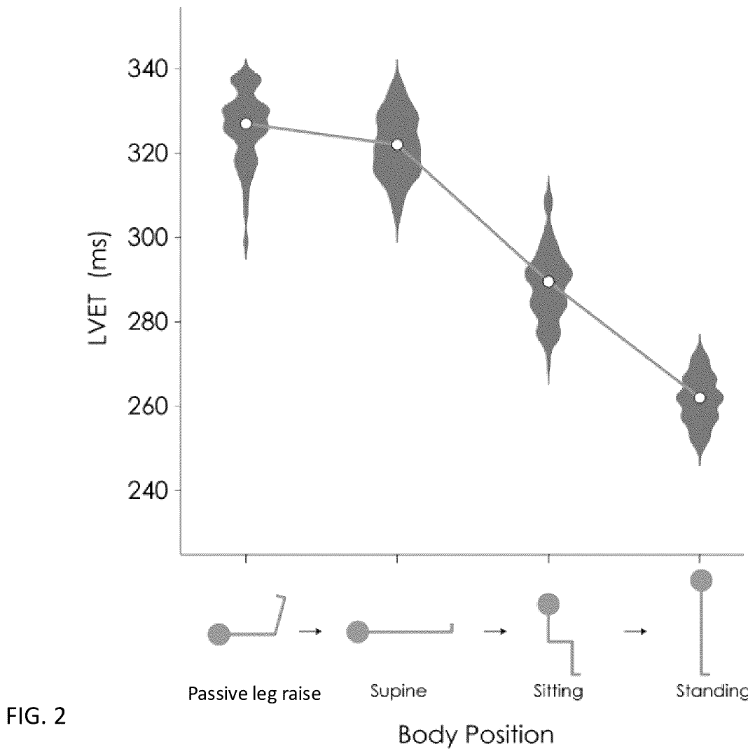

The influence of increasing the preload of the heart is well illustrated in FIG. 2. As the patient moved from the standing to sitting to supine to passive leg raise positions, the venous return to the heart increased. In a normal heart, the resulting increase in blood volume creates an increase in stroke volume and LVET. The change from standing to supine creates the largest change in LVET. The change in effective circulating volume is commonly estimated to be around 300 mL, as blood moves from the lower extremities to the thorax. The change in LVET with a passive leg raise is less than that from standing to supine, as the volume change is less.

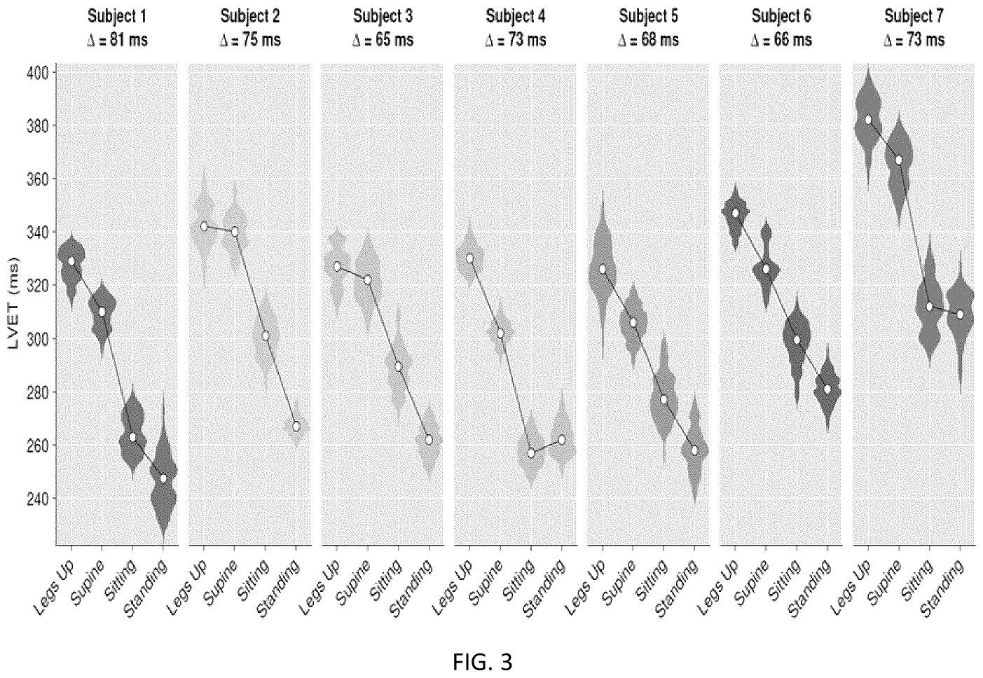

FIG. 3 illustrates the results obtained from several subjects of varying heights. The change in LVET due to positional changes has some variance due to different leg lengths and sizes but the LVET change is always an increase as the amount of filling pressure is increased.

A change in preload, such as that induced by passive leg raise (PLR), positional changes, or other preload-modifying maneuvers, serves as a functional test of myocardial performance by assessing the heart's response to a repeatable physiological stimulus, rather than relying on single-point measurements. The repeatability of the test is effectively “built-in”; as movement between posture positions is highly repeatable. Additionally, patient height and vascular dimensions remain constant over time. In a normally functioning heart, or in aortic stenosis (AS) without significant myocardial impairment, increased preload results in greater end-diastolic volume and stroke volume, leading to a measurable increase in left ventricular ejection time (LVET) via the Frank-Starling mechanism. In contrast, in the presence of myocardial dysfunction, the left ventricle is unable to augment contractility in response to increased preload, resulting in a blunted or absent increase in LVET, stroke volume, or cardiac output, and a diminished Frank-Starling response. This diminished physiological response provides a non-invasive and reproducible marker of impaired myocardial reserve or function.

A static, point-in-time measurement (often called a “resting measurement”) captures an absolute LVET value at a single moment and, when repeated, yields a trend that is vulnerable to baseline drift, sympathetic tone fluctuations, and patient stress. In contrast, a dynamic—or provocative—test introduces a controlled preload challenge (for example, a passive leg-raise) that reproducibly perturbs venous return. By recording LVET and related systolic time intervals, stroke volume, and cardiac output immediately before and after that disturbance, the system performs a delta analysis—a within-test comparison of relative change that is largely insensitive to small baseline variances. The use of a dynamic functional test enables the quantification of reductions in contractile reserve and blunted Frank-Starling responsiveness by minimizing the impact of baseline variances and differences in general physiological state.

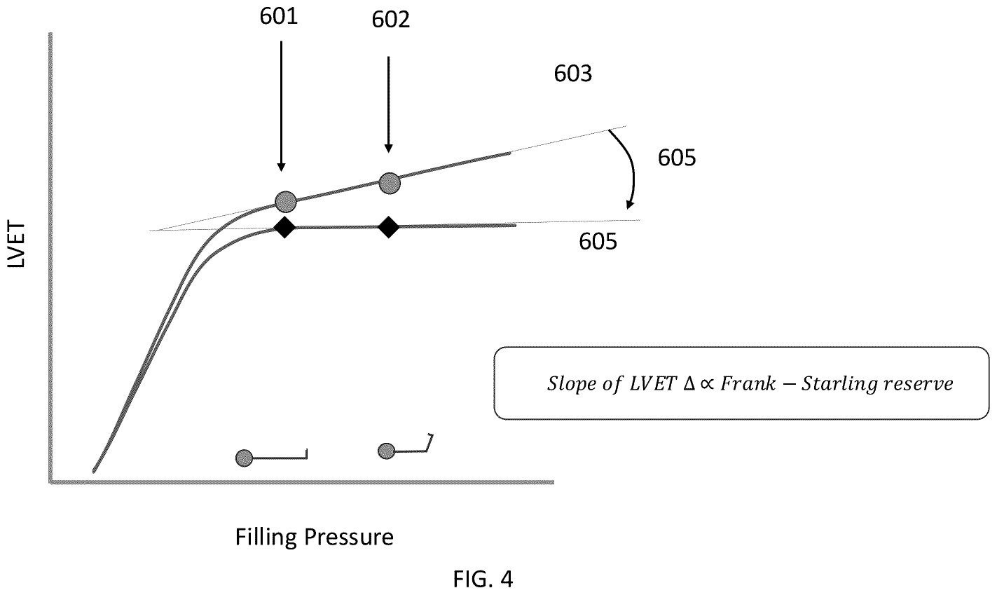

FIG. 4 illustrates the concept of such a functional test and the impact of diminished Frank-Starling response over time for a patient who does not have AS. The illustration shows the LVET with the patient in a supine position (601) and a second measurement when the patient has increased preload via a passive leg raise (602). The circle points illustrate a normal Frank-Starling response with an increase in LVET. In contrast, the diamond points illustrate a diminished Frank-Starling response with no increase in LVET. Line 603, the straight line through the two circle markers, has a steep slope, reflecting a large ΔLVET in response to increased preload and thus preserved myocardial function. By contrast, line 604, the line through the two diamond markers, is markedly flatter, indicating a minimal LVET change with the same preload increase and revealing blunted Frank-Starling responsiveness.

### Functional Testing with Aortic Stenosis

In patients with aortic stenosis (AS), the aortic valve area is reduced, increasing the resistance against which the left ventricle must eject blood. With normal myocardial function, the increased afterload prolongs LVET because the left ventricle needs more time to overcome the higher resistance and maintain cardiac output.

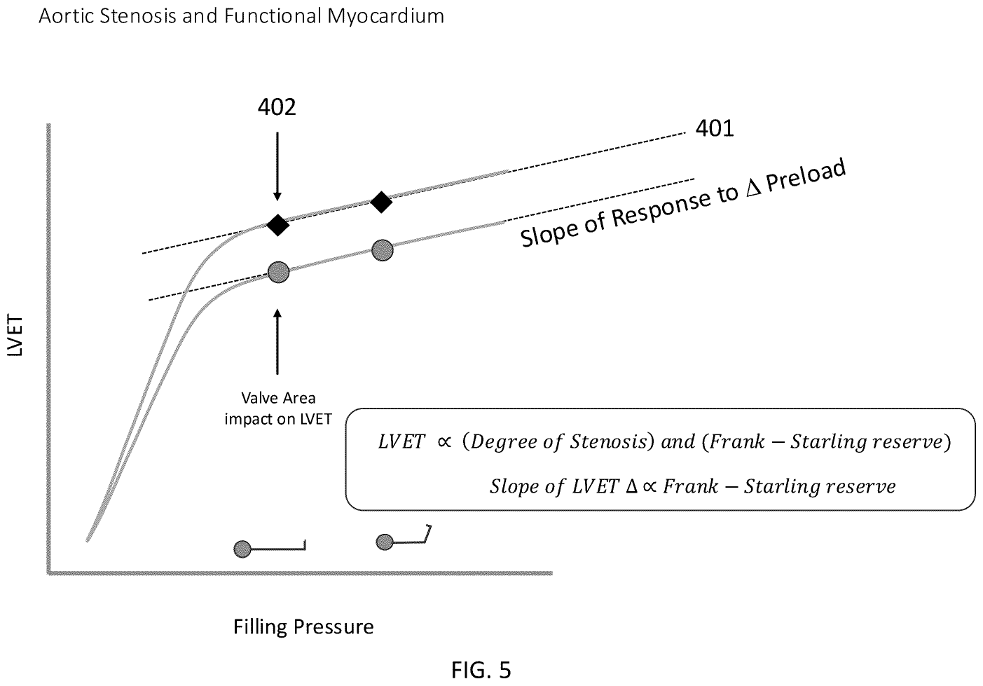

FIG. 5 illustrates the impact of aortic stenosis on the LVET-filling-pressure relationship while preserving Frank-Starling reserve. The two solid curves plot LVET versus filling pressure for hearts with the same contractile capacity but different valve areas. Circle markers denote LVET before and after a passive leg-raise in the non-stenotic heart (normal valve area), and diamond markers denote the corresponding points in the stenotic heart (reduced valve area). The vertical separation between the circle and diamond curves at equivalent filling pressures (arrow 402) quantifies the LVET elongation caused by decreased valve area (AS increases LVET). Despite this baseline shift, the dashed lines 401 and 403, each drawn through the pre- and post-challenge markers for their respective curves, have identical slopes, indicating myocardial function remains preserved as evidenced by the maintenance of ΔLVET/Δpreload, irrespective of valve obstruction. The maintenance of a consistent slope, observed across time-separated measurements, is a measure of functional myocardium. Thus, while the vertical offset measures the degree of aortic stenosis (assuming no other cardiac alterations), the within-test delta analysis isolates true contractile reserve and Frank-Starling responsiveness independently of valvular changes

### Functional Testing with Aortic Stenosis and Decreasing Left Ventricular Function

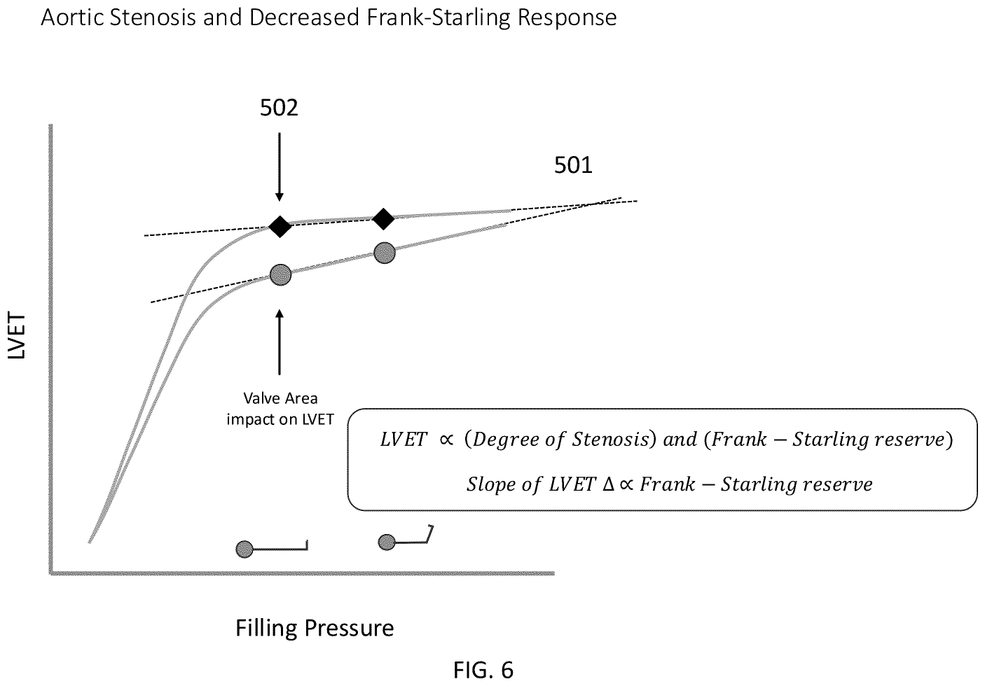

FIG. 6 depicts a patient with isolated aortic stenosis and preserved myocardial function, as evidenced by an elevated resting LVET (diamond marker offset 502) and a robust Frank-Starling response to passive leg-raise (steep slope 501). Prolonged pumping through the narrowed valve, however, imposes chronic pressure overload that eventually injures the myocardium. FIG. 6 illustrates this early damage: the resting LVET at supine baseline remains elevated by the same offset (502), but the PLR-induced prolongation of LVET is markedly attenuated, yielding a significantly flatter response line (slope 501). The combination of an unchanged vertical offset with a blunted ΔLVET/Δpreload thus provides direct, within-test evidence of declining Frank-Starling reserve.

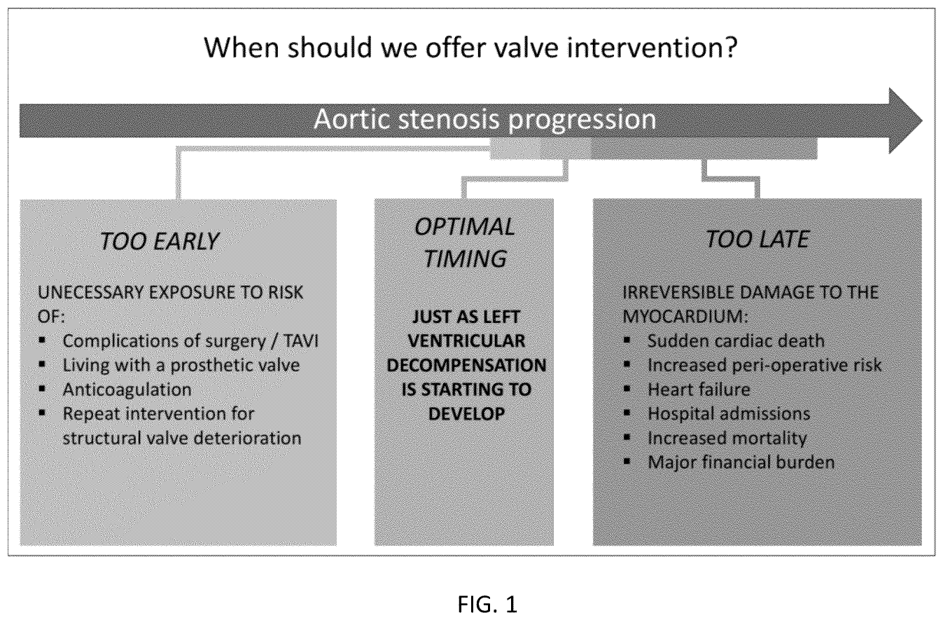

The persistence of elevated resting LVET alongside loss of preload-induced prolongation marks the earliest detectable loss of Frank-Starling reserve. At this transition, the ventricle can no longer recruit additional stroke volume when challenged, signaling the onset of irreversible myocardial deterioration. Identifying this precise shift, from preserved to blunted Frank-Starling responsiveness, defines the optimal window for valve replacement, balancing the risks of premature intervention against the consequences of delayed treatment (see FIG. 1).

### Further Disease Progression

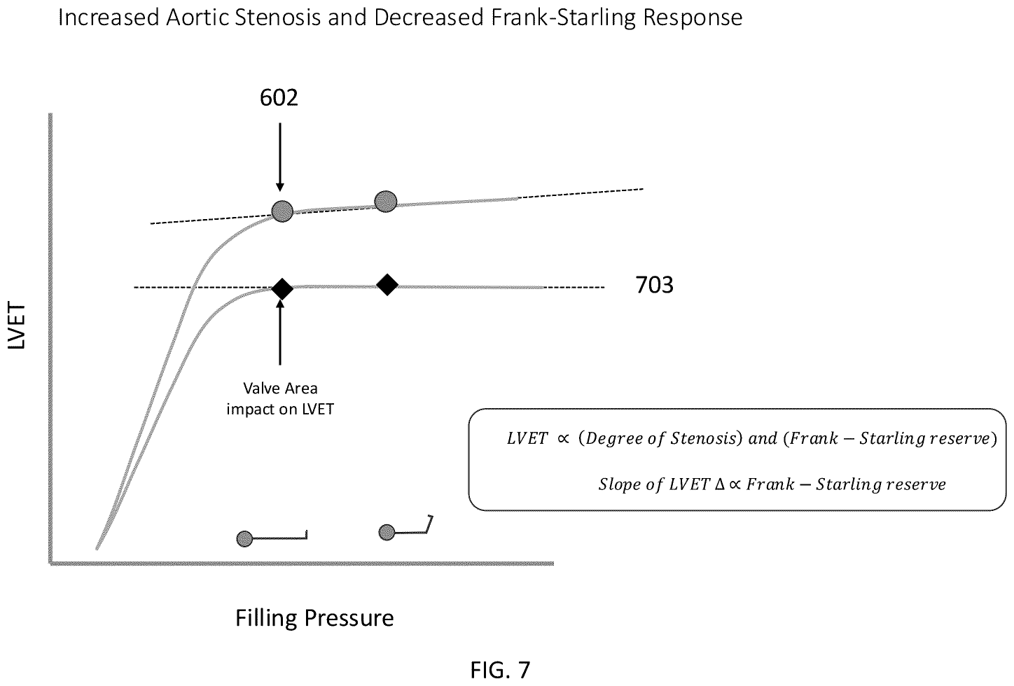

FIG. 7 demonstrates further disease progression. The ongoing pressure overload through the stenotic valve has inflicted irreversible myocardial injury. Relative to FIG. 6, the supine-baseline LVET has declined from its earlier state, circle marker, to a lower diamond marker (702), signifying that even resting ejection performance has deteriorated. The PLR-induced response line (703) not only remains flat, reflecting persistent loss of Frank-Starling reserve, but is now also shifted downward compared to the preserved slope seen in FIG. 5 and the early blunted response in FIG. 6. This combined vertical and angular displacement indicates that the ventricle can neither sustain prolonged ejection time at rest nor recruit additional stroke volume under increased preload. By this stage, myocardial contractile capacity is severely compromised, and the window for valvular intervention has narrowed. Aortic valve replacement will confer markedly less functional recovery than intervention timed at the initial loss of preload responsiveness.

### SUMMARY

The invention provides a diagnostic method and system for determining the optimal timing of aortic valve replacement by capturing cardiac function metrics—such as systolic time intervals (e.g., LVET) and cardiac output—via a simple, noninvasive instrumentation pathway. In isolation, AS typically prolongs LVET because the left ventricle requires more time to eject blood through a narrowed valve, whereas myocardial dysfunction shortens LVET due to impaired contractile efficiency and the inability to sustain ejection. Because these two mechanisms exert opposing effects on LVET, a single measurement taken at rest cannot reliably distinguish valvular from myocardial contributions. By first measuring baseline LVET—which reflects the combined effects of aortic stenosis and intrinsic myocardial contractility—and then applying a defined preload perturbation (e.g., passive leg-raise) to perform a within-test ΔLVET/Δpreload analysis, the system separately quantifies valvular obstruction (vertical LVET offset) and Frank-Starling reserve (slope of LVET change). This elegant, repeatable diagnostic paradigm isolates true contractile capacity—independent of valve area—and identifies the earliest transition from compensated to decompensated myocardium, thereby guiding the precise window for valve intervention.

### Measurement and Measurement Metrics

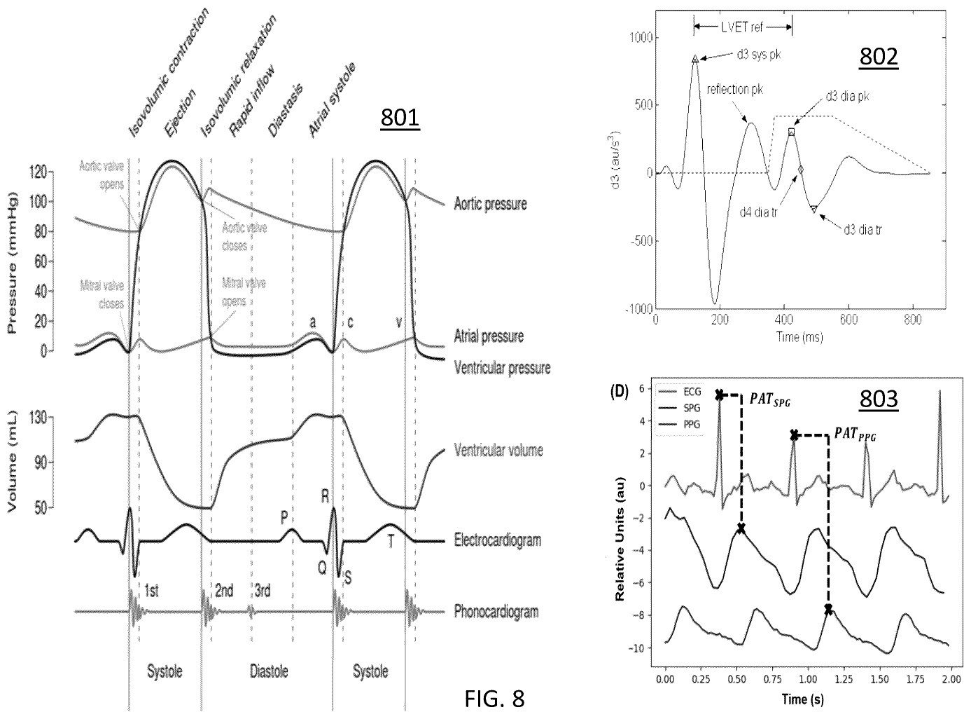

A variety of measurement technologies and associated physiological metrics can be employed to measure the ventricular response to a defined, reversible increase in preload. The change observed in each metric can be plotted against the known preload increment to reconstruct an individual patient's Frank-Starling slope. Physiological metrics can be broadly divided into two scientific categories. The first class is fundamentally based on systolic-time intervals (STIs), the electrical and mechanical events that occur during the cardiac cycle. FIG. 8 is an illustration of systolic time intervals, shown as a composite waveform diagram that overlays aortic, ventricular, and atrial pressure curves with ventricular volume, ECG deflections, and phonocardiographic heart-sound markers to portray the successive phases of the cardiac cycle. The second class captures preload responsiveness without explicit timing, relying instead on beat-to-beat changes in flow, volume, or waveform amplitude. Each class is described in conjunction with the measurement systems that generate the underlying data.

Examples of fiducial marks are illustrated in FIG. 8. Chart 801 shows multiple inflection points associated with pressure, electrical activity, and sound. Chart 802 is a derivative of a PPG waveform and shows many fiducial marks derived from the derivative. Chart 803 provides a comparison between ECG, PPG and SPG.

### STI-Centered Metrics.

When the heart experiences an acute increase in venous return, a healthy ventricle lengthens its left-ventricular ejection time (LVET) and shortens its pre-ejection period (PEP); the resulting change in the PEP/LVET or ICT/LVET ratio is therefore a compact marker of preserved contractile reserve. Other useful STI indices include the isovolumic-contraction time, the electromechanical activation time, and the composite QS2 interval. STI sensing methods include: optical photoplethysmography and speckle plethysmography, radar plethysmography, phonocardiography, seismocardiography, ballistocardiography, and the axial or radial acceleration peaks captured by a chest-mounted inertial unit. Because STIs are time-based differential measurements, rather than absolute measurements, they require no calibration.

### Non-STI Metrics.

A complementary set of measurements characterizes Frank-Starling responsiveness without relying on timing. Thoracic bioimpedance, as defined herein, computes stroke volume and cardiac output from beat-to-beat changes in chest wall impedance; a ten-percent rise in impedance-derived stroke volume after the preload maneuver is widely accepted as evidence of preserved reserve. Bio-reactance interrogates the phase shift of the same injected current. Gyrocardiography records the rotational momentum of ventricular ejection with a micro-electromechanical gyroscope; the amplitude of the primary systolic crest increases when preload is successfully recruited. A wearable Doppler patch emits and receives continuous-wave or pulsed ultrasound from a superficial artery, integrates the velocity-time product of each beat, and reports the corrected flow time, which prolongs by roughly fifteen milliseconds in the fluid-responsive ventricle. Additional non-STI indices—pulse-pressure variation, stroke-volume variation, pleth variability index, or the velocity-time integral obtained by hand-held echocardiography—can also be used.

Raw sensor waveforms, together with the positional cues that verify the preload maneuvers, are synchronized, reduced to numerical parameters (e.g., Δ-LVET, Δ-stroke volume, Δ-corrected flow time). Those parameters are provided to the data processing system and compared across the baseline and post-maneuver periods to quantify an individual patient's Frank-Starling responsiveness.

### Instrumentation Embodiments

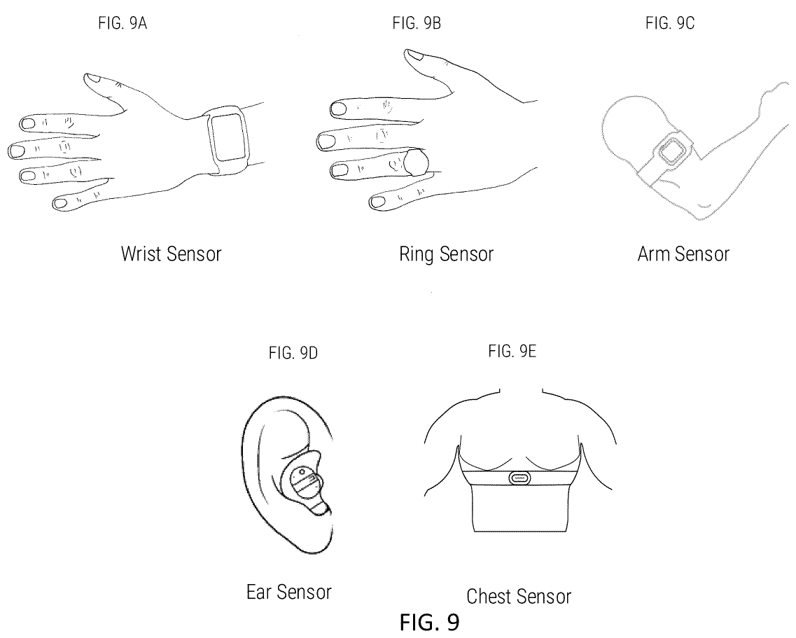

The present invention supports a variety of sensor types and configurations capable of capturing a Frank-Starling response via systolic time intervals, particularly left ventricular ejection time (LVET)—using many modalities to including optical modalities such as photoplethysmography (PPG) and speckle plethysmography (SPG). FIG. 9 illustrates representative hardware embodiments showing several potential sensor placements, including wrist (9A), ring/finger (9B), upper arm (9C), ear (9D), and chest (9E). Several configurations are compatible with both reflective and transmissive optical measurement approaches and may be used individually or in combination to enhance signal robustness.

Each embodiment enables static physiological measurements taken in distinct postural states—for example, one recording in a supine position and a second after a preload-increasing maneuver such as a passive leg raise (PLR). The diversity of sensor placements provides flexibility in accommodating different user needs, body types, and clinical contexts.

### Types of Passive Leg Raises

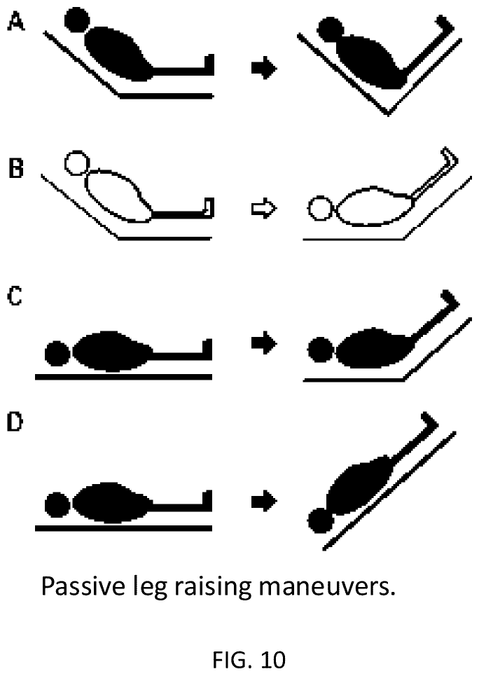

FIG. 10 illustrates a series of passive leg raising maneuvers used to increase venous return to the heart by repositioning the patient. Each column (A-D) depicts a distinct initial posture (e.g., semi-recumbent, upright, supine) followed by a corresponding leg elevation. These maneuvers are designed to induce a transient and reversible increase in preload, allowing for the functional testing of Frank-Starling reserve without the need for pharmacological or fluid-based challenges. The most common PLR is identified with the letter C and is easily performed on most examination tables by raising the lower legs to 45 degrees with a segment of the table or a wedge pillow.

### Measurement Sequence

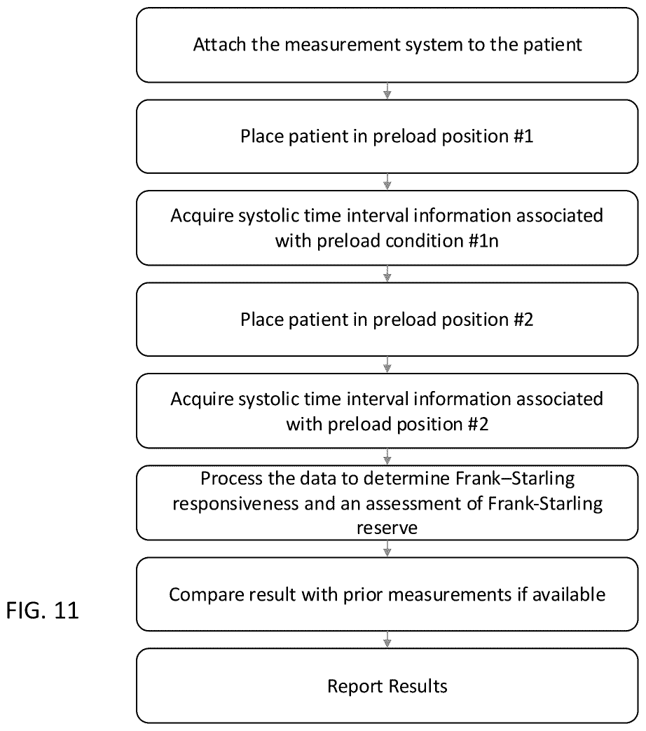

FIG. 11 illustrates an example measurement sequence. The sequence begins with the subject placed in a baseline supine position. Once the subject is at rest and hemodynamically stable, an initial optical recording is acquired using a measurement system as defined in FIG. 9. This first measurement captures the left ventricular ejection time (LVET) under resting preload conditions.

Next, a controlled preload modification is introduced by passively elevating the subject's legs, commonly using a wedge or leg support, to increase venous return. After allowing a brief equilibration period to ensure circulatory stability, a second measurement is taken in the new posture using the same sensor system.

The two recordings are then compared to evaluate the change in LVET in response to the preload increase. This delta analysis enables the system to determine whether a Frank-Starling response is present and proved information regarding the functional reserve of the myocardium.

### Example Measurement Embodiment

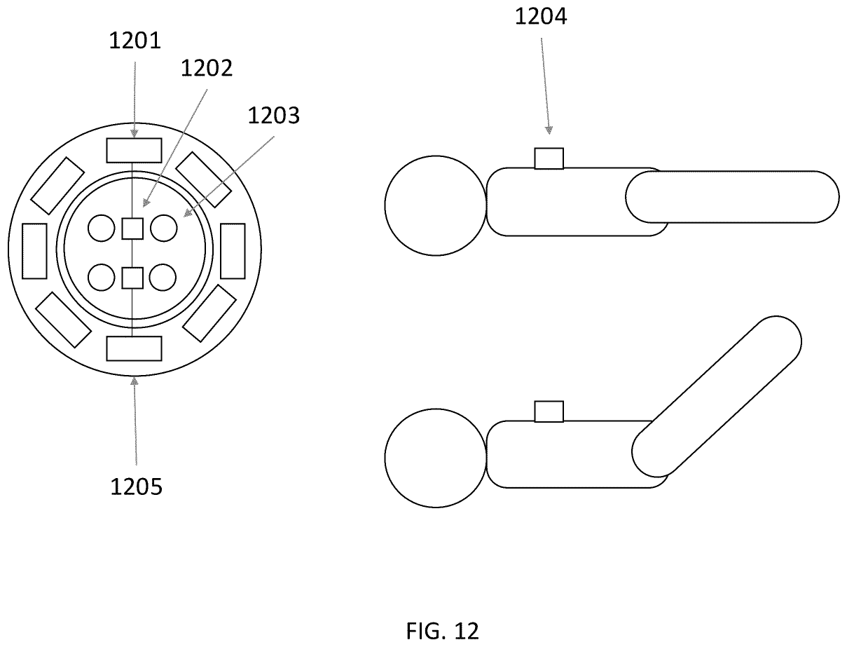

FIG. 12 illustrates a combined photoplethysmography (PPG) and speckle plethysmography (SPG) measurement system configured for chest-based acquisition of cardiovascular signals. The circular module on the left depicts the sensor head, which integrates both coherent and incoherent light sources along with a shared detector array. Coherent light sources, such as laser diodes, 1202, are used to generate speckle patterns for SPG measurements, while incoherent sources, 1203 such as light-emitting diodes (LEDs), provide illumination for PPG signal acquisition. The typical operating wavelengths for both systems are in the visible and near-infrared ranges, spanning from 470 nm to 940 nm. A central detector array 1201 captures both types of optical signals, enabling robust characterization of blood flow and volume dynamics from a single anatomical site.

In some embodiments, the measurement unit includes an inertial measurement unit (IMU), 1205, gyroscope, or accelerometer to detect and record the orientation and movement of the sensor during use. This capability ensures that data acquisition occurs only under stable, predefined postural conditions (e.g., supine, standing, or passive leg raise) and enables verification that the patient has fully transitioned into the intended position before measurement begins. Additionally, motion artifacts can be identified and excluded, improving signal quality and enhancing the reliability of systolic time interval measurements such as LVET.

The right side of the figure shows the sensor system applied to a subject, 1204, in two distinct postural conditions. In the upper diagram, the subject is positioned supine with legs extended, while the lower diagram shows the same subject after passive leg elevation to increase venous return. In both states, the chest-mounted sensor remains fixed in place, allowing stable, motion-free data acquisition once each posture is achieved.

### Data Pathway

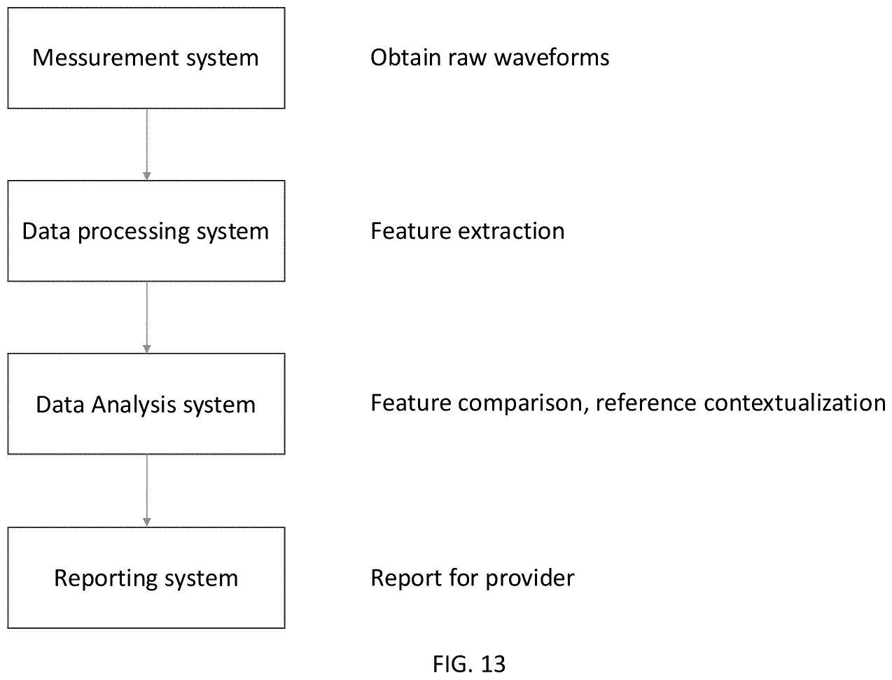

FIG. 13 is an example data pathway. Significant elements of the pathway include:

Measurement system—Sensors (e.g., PPG, Doppler patch, gyroscope) and the inertial unit acquire raw physiological waveforms plus posture status.

Data-processing system—The raw streams are cleaned, time-aligned, and converted into numerical metrics such as Δ-LVET or Δ-stroke volume.

Data-analysis system—Those metrics are compared with baseline values and reference thresholds to decide whether the ventricle is preload-responsive or reserve-limited.

Reporting system—The classification and supporting numbers are formatted into a concise report for clinician or patient review.

### Data Processing

In the Measurements and Measurement Metrics section, a variety of sensing modalities and physiological parameters were presented. The following section provides one example embodiment of the processing and analysis workflow for a simple PPG-based measurement. While PPG is used here as a representative example, it will be recognized by those skilled in the art that similar and related processes could be applied to other measurement methods (e.g., SPG, phonocardiography, seismocardiography) and other physiological metrics (e.g., pulse transit time, stroke volume, cardiac output).

The data processing system, for this example, refers to a computational module—hardware, firmware, or software, that transforms raw photoplethysmography (PPG) signals into clean, interpretable physiological metrics, including but not limited to heart rate (HR), interbeat interval (IBI), and left ventricular ejection time (LVET). This system operates autonomously or semi-autonomously as part of the measurement apparatus or via a connected computing platform.

Upon initiation, the system begins data acquisition, as described in FIG. 11, by recording optical waveforms from the PPG sensor. These signals are captured at a sampling rate to preserve the temporal fidelity of the pulse morphology. Simultaneously, an onboard inertial measurement unit (IMU) records device motion and orientation to provide contextual information for signal quality assessment.

The raw PPG waveform then undergoes preprocessing, which includes filtering to remove high-frequency noise (e.g., due to ambient light or electronic interference), baseline drift correction, and smoothing to enhance waveform clarity. Motion artifacts are identified using both signal morphology (e.g., abrupt discontinuities, irregular peaks) and IMU data. Epochs of data associated with active movement or unstable positioning are flagged and excluded from further analysis.

In addition to motion, the processing system evaluates pulse quality by analyzing beat-to-beat variability, amplitude, and saturation. Saturated signals, due to poor contact or sensor overexposure, are discarded. Individual beats are then segmented using peak detection algorithms, and each valid pulse is assessed for consistency with neighboring pulses and expected physiological ranges.

For those pulses deemed acceptable, the system extracts relevant physiological features. This includes calculating the left ventricular ejection time (LVET), defined as the duration from pulse onset to dicrotic notch or inflection point, alongside HR and waveform shape consistency. The LVET is typically obtained by using the second derivative of the PPG waveform. These features are averaged across a validated time window to yield a representative value for the subject's cardiovascular state in that specific posture (e.g., supine or during passive leg raise).

This process is repeated for each postural condition, ensuring that both measurements are derived from clean, high-quality data acquired in stable physiological states. The final output is a pair of LVET values, one for each condition, which can be further analyzed to evaluate preload responsiveness and myocardial reserve. It is recognized that the details of the processing would be different for impedance cardiography, but the objective of obtaining reliable, valid data is the same.

### Data Analysis and Reporting System

The data analysis system interprets processed physiological data and generates diagnostic or comparative insights. The following section provides one example embodiment of the processing and analysis workflow for a simple PPG-based measurement. Upon receiving the PPG derived LVET and related metrics from two postures, the system computes the absolute and relative change in LVET (ΔLVET and % ΔLVET), representing the patient's Frank-Starling response. If prior measurement data exists for the same patient, the system performs a longitudinal comparison, identifying trends in resting LVET and changes in preload responsiveness over time.

In the absence of historical data, the analysis module compares the patient's values to population norms, possibly stratified by age, sex, and cardiovascular risk. Reference ranges for expected LVET and preload-induced LVET changes are used to flag deviations suggestive of either elevated afterload (prolonged resting LVET) or blunted myocardial reserve (reduced ΔLVET).

The reporting module compiles these results into a clinical decision support report, which may include graphical plots (e.g., LVET vs. filling pressure curves), tabular data, and interpretive summaries. These outputs help clinicians identify early signs of decompensation, monitor disease progression, and determine the optimal timing for aortic valve replacement or additional diagnostic evaluation.

### Examples of Reported Results

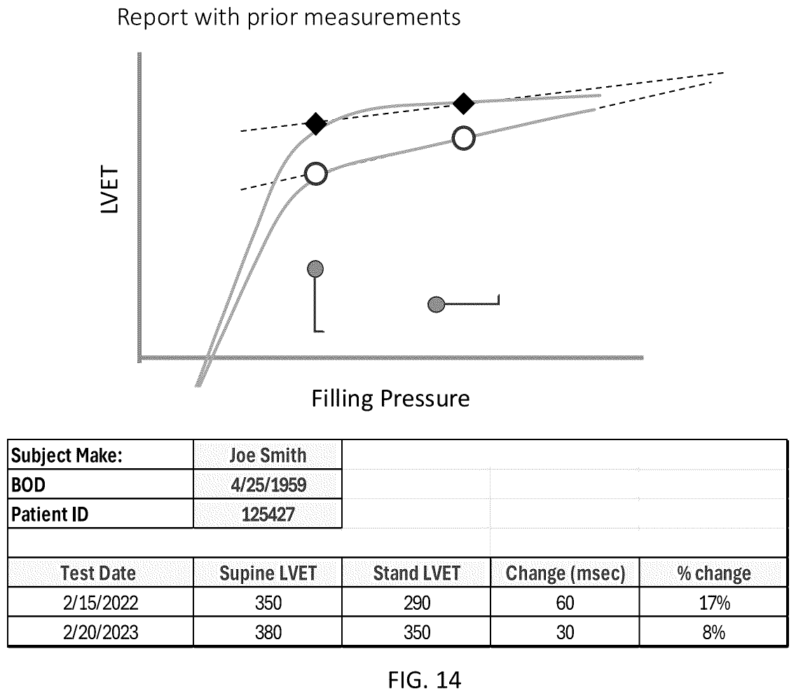

FIG. 14 is an illustration of a Report with Prior Measurements (Supine to Standing):

This report presents longitudinal data for a patient, including previous measurements, allowing for intra-subject comparison over time. The primary indicator of aortic stenosis progression is the increase in supine LVET, which rose from 350 ms to 380 ms between 2022and 2023. This increase suggests heightened afterload, consistent with worsening valve obstruction. Concurrently, the patient exhibits a decrease in the preload responsiveness; the absolute change in LVET from standing to supine decreased from 60 ms to 30 ms, and the relative change dropped from 17% to 8%. This attenuation shows a decline in Frank-Starling reserve. Together, these findings point toward both advancing valvular disease and early myocardial dysfunction. The combination of prolonged baseline LVET and diminished preload-induced LVET augmentation strongly supports consideration for aortic valve replacement (AVR).

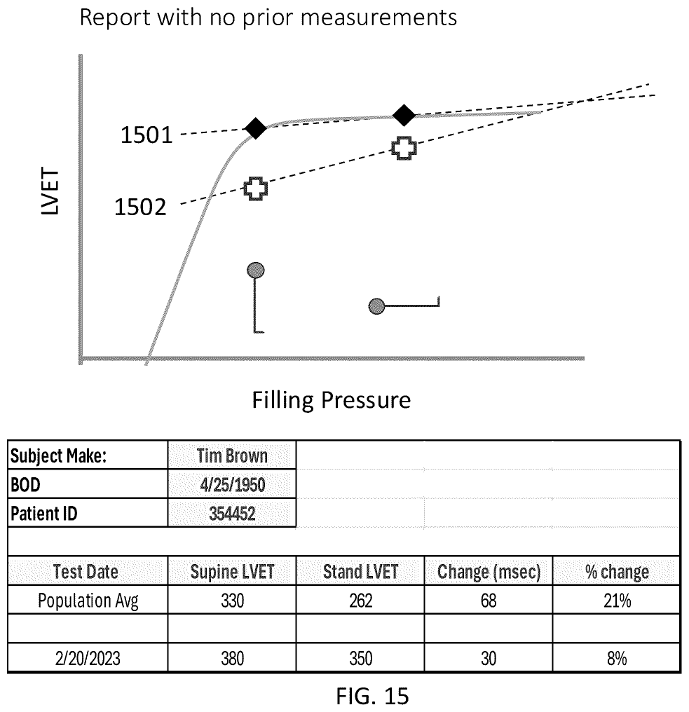

FIG. 15 is an illustration of a Report Without Prior Measurements (Population Comparison):

This report assesses a subject without prior personal baseline data. Interpretation relies on population-based norms. The subject demonstrates an elevated supine LVET, consistent with increased resistance to ejection likely due to aortic stenosis. The change in LVET after preload enhancement (e.g., via supine position) is notably reduced, 1501, relative to the population matched results, 1502, demonstrating a blunted Frank-Starling response. This pattern mirrors that seen in FIG. 14 and similarly indicates the coexistence of significant aortic outflow obstruction and declining Frank Starling reserve. Although progression over time cannot be assessed, the findings support a diagnosis of AS with emerging ventricular dysfunction and warrant close clinical evaluation for potential intervention.

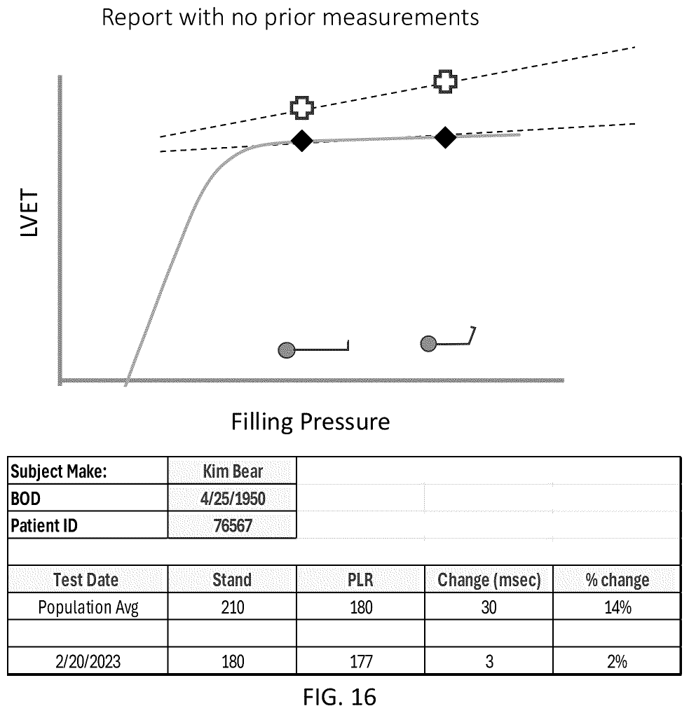

FIG. 16 is an illustration of a Report Without Prior Measurements (Supine to PLR):

In this case, the subject exhibits a low baseline LVET relative to population norms and shows minimal or no increase in LVET following a passive leg raise. This flat Frank-Starling curve is characteristic of severe myocardial dysfunction, indicating that the left ventricle cannot meaningfully increase stroke volume despite increased preload. However, unlike the previous cases, the low resting LVET provides no direct evidence of aortic stenosis as there are many causes of a decrease in LVET. Therefore, while the pattern confirms impaired myocardial reserve, the underlying etiology cannot be definitively determined from this report alone. The dysfunction could be due to prior myocardial infarction, cardiomyopathy, or advanced AS, but further diagnostic workup (e.g., echocardiography, valve imaging) would be required to clarify the cause. The decreased LVET and lack of preload response suggest significant myocardial damage. If this patient has AS, the benefit of replacement relative to the subject in FIG. 14 would be diminished.

### Determining the Lower Bound of a “Normal” Physiological Response

Population-derived data can serve as a powerful foundation for defining the lower bounds of normal physiological responses to preload-modifying maneuvers. As illustrated in the accompanying table, one method involves aggregating systolic time interval responses—such as changes in LVET during transitions from legs-up to supine, supine to sitting, and supine to standing—across a reference group of healthy subjects. By calculating the average response and its associated standard deviation, it becomes possible to set quantitative thresholds (e.g., mean minus one standard deviation) that reflect the lower bound of expected Frank-Starling responsiveness. This boundary helps delineate what constitutes a “normal” response and flags values that may indicate impaired myocardial reserve.

This approach can be further refined by stratifying the reference population to match the individual subject under evaluation across key demographic and clinical variables—such as age, sex, body size, baseline LVET, or comorbidities. A 70-year-old male, for example, might be compared not to a general population but to a subgroup of demographically similar peers. This contextualization enhances specificity and allows more personalized interpretation of deviations from normal.

Several variants of this methodology are feasible. One could use raw differences in LVET (e.g., ≥9.2 ms increase from legs-up to supine as a minimum threshold), or apply percent change criteria (e.g., ≥2.8% increase), depending on whether absolute or relative sensitivity is preferred. Additionally, thresholds can be anchored to single transitions (such as ΔPLR), or extended across multiple transitions to generate a composite preload-responsiveness profile. Machine learning techniques could also be deployed to identify nonlinear patterns or interactions that improve predictive value.

Importantly, the degree of abnormality can also be scaled in terms of statistical deviation. A response falling more than one standard deviation below the population mean may signal a mildly impaired response, while deviations exceeding two standard deviations may indicate a profoundly abnormal Frank-Starling reserve, suggestive of more advanced myocardial dysfunction. This gradation allows for a more nuanced clinical interpretation—flagging subtle dysfunction in early stages and distinguishing it from more significant hemodynamic compromise.

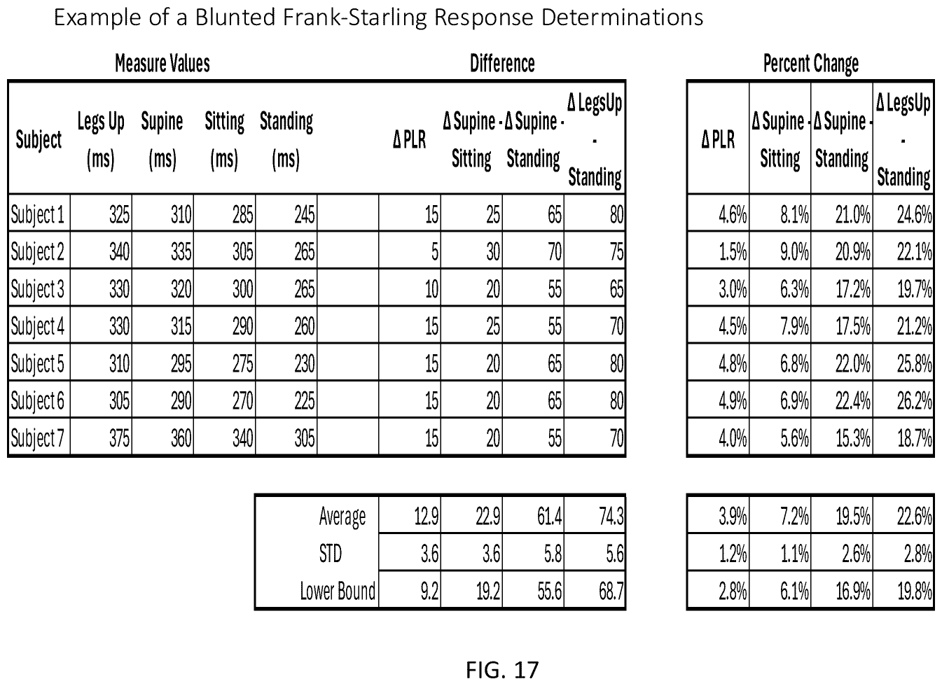

It is also important to recognize that not all postural transitions impose the same physiological load or create equivalent myocardial stretch. For example, a subject might demonstrate a normal or near-normal LVET response during a supine-to-sitting or supine-to-standing transition, yet exhibit an abnormal response during a passive leg raise (PLR). This discrepancy can occur because PLR uniquely increases venous return by gravitationally shifting pooled blood from the lower extremities toward the central circulation, thereby inducing a more substantial and immediate increase in preload. In essence, PLR serves as a maximal stretch test of myocardial reserve—challenging the ventricle at its upper limit of physiological stretch. If the heart cannot augment contractility under this condition, it reveals a limitation that might not be apparent with more modest preload shifts. Consequently, PLR offers a more sensitive and provocative assessment of the Frank-Starling mechanism than other posture-based tests. FIG. 17, derived from the data from FIG. 3, illustrates the process of using measured data to define appropriate thresholds.

Ultimately, this population-based framework enables a data-driven method for detecting subclinical cardiac dysfunction before overt symptoms or structural changes emerge, making it a valuable tool for proactive cardiovascular risk stratification and decision support in clinical care.

Those skilled in the art will recognize that the present invention can be manifested in a variety of forms other than the specific embodiments described and contemplated herein. Accordingly, departures in form and detail can be made without departing from the scope and spirit of the present invention as described in the appended claims.

While the concepts of the present disclosure are susceptible to various modifications and alternative forms, specific embodiments thereof have been shown by way of example in the drawings and described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described can include a particular feature, structure, or characteristic, but not every embodiment must necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments can be implemented, in some cases, in hardware, firmware, software, or any combination thereof. The disclosed embodiments can also be implemented as instructions carried by or stored on a transitory or non-transitory machine-readable (e.g., computer-readable) storage medium, which can be read and executed by one or more processors. A machine-readable storage medium can be embodied as any storage device, mechanism, or other physical structure for storing or transmitting information in a form readable by a machine (e.g., a volatile or non-volatile memory, a media disc, or other media device).

In the drawings, some structural or method features are shown in specific arrangements and/or orderings. However, it should be appreciated that such specific arrangements and/or orderings might not be required. Rather, in some embodiments, such features can be arranged in a different manner and/or order than shown in the illustrative figures. Additionally, the inclusion of a structural or method feature in a particular figure is not meant to imply that such feature is required in all embodiments and, in some embodiments, might not be included or might be combined with other features.

What is claimed is:

1. A non-invasive cardiac assessment apparatus comprising:
(a) an optical sensor module configured to generate a physiological waveform that exhibits (i) a first fiducial point corresponding to aortic-valve opening and (ii) a second fiducial point corresponding to aortic-valve closing;
(b) an inertial-measurement unit (IMU) configured to verify (a) a first body position and (b) a second body position that differs from the first by a gravity-mediated increase in venous return;
(c) at least one processor, operatively coupled to the sensor module and the IMU and programmed to
(i) derive a first left-ventricular-ejection-time value LVET₁ from the waveform acquired in the first body position,
(ii) derive a second left-ventricular-ejection-time value LVET₂ from the waveform acquired in the second body position,
(iii) compute a preload-response metric ΔLVET=LVET₂−LVET₁, and
(iv) compare the preload-response metric with a Frank-Starling-reserve threshold to classify the myocardium as (i) normal Frank-Starling response or (ii) diminished Frank-Starling response; and
(d) an output report configured to present the measurement information.

2. The apparatus of claim 1, wherein the sensor module comprises a reflective photoplethysmography emitter-detector pair operating at a wavelength between 770 nm and 940 nm.

3. The apparatus of claim 1, wherein the sensor module comprises a speckle plethysmography sensor including a coherent light source and an imaging detector.

4. The apparatus of claim 1, wherein the sensor module and the IMU are co-located in a finger-worn ring housing and the sampling modality is transmission based.

5. The apparatus of claim 1, wherein the first body position is supine and the second body position is a passive leg raise of 30°-60° relative to horizontal.

6. The apparatus of claim 1, wherein the Frank-Starling-reserve threshold is met when ΔLVET fails to exceed 15% of LVET when the preload change is a stand-to-supine transition.

7. The apparatus of claim 1, wherein the processor is further programmed to update the Frank-Starling-reserve threshold adaptively using longitudinal ΔLVET data from the same patient.

8. A computer-implemented method of identifying a transition to irreversible myocardial damage in a patient with suspected aortic stenosis, the method comprising:
(a) acquiring a first physiological waveform while the patient is in a baseline posture;
(b) deriving a first left-ventricular-ejection-time value LVET₁ from the first waveform;
(c) using inertial sensing to confirm that the patient has assumed a preload-augmenting posture that increases venous return;
(d) acquiring a second physiological waveform while the patient is in the preload-augmenting posture;
(e) deriving a second left-ventricular-ejection-time value LVET₂ from the second waveform;
(f) computing ΔLVET=LVET₂−LVET₁;
(g) comparing ΔLVET with a Frank-Starling-reserve threshold obtained from at least one of (a) a fixed value, (b) a population-matched model, or (c) a prior measurement of the same patient; and
(h) outputting a report containing the LVET₁, LVET₂, and ΔLVET.

9. The method of claim 8, wherein the preload-augmenting posture is a passive leg raise that elevates the patient's legs to at least 45 degrees above horizontal.

10. The method of claim 8, further comprising averaging each LVET value over at least five consecutive cardiac cycles.

11. A non-transitory computer-readable storage medium storing instructions that, when executed by one or more processors, cause the processors to perform the method of claim 10.
